(19) United States
(12) Patent Application Publication
(10) Pub. No.: US 2010/0035240 A1
(43) Pub. Date: Feb. 11, 2010

(54) METHODS AND KIT FOR THE PROGNOSIS OF BREAST CANCER

(75) Inventor: Wen Guo Jiang, Heath Park (GB)

Correspondence Address: KING & SCHICKLI, PLLC, 247 NORTH BROADWAY, LEXINGTON, KY, 40507, US

(73) Assignee: Cardiff Biologicals Limited

(21) Appl. No.: 11/573,487

(22) PCT Filed: Jul. 27, 2005
(86) PCT No.: PCT/GB2005/002971

§ 371 Date: Oct. 20, 2009

(30) Foreign Application Priority Data
Aug. 10, 2004 (GB) 0417740.8
Dec. 7, 2004 (GB) 0426777.9

(51) Int. Cl. C12Q 1/68; C40B 40/08; C07H 21/04

(52) U.S. Cl. 435/6; 506/17; 536/24.3

(57) ABSTRACT

The present invention relates to a method and kit, including parts thereof, for the prognosis of breast cancer. In particular, the method involves identifying a gene expression pattern, or molecular signature, that indicates the likelihood of survival of a patient with breast cancer, and/or likelihood of recurrence of the disease in a patient being treated, or having been treated, for breast cancer, and the likelihood of a patient having a metastatic form of cancer. Six molecular signatures, comprising twelve groups/sets of molecular markers have been identified, which have relevance in determining the prognosis of a given breast cancer. Each molecular signature comprises a plurality of genetic markers whose expression, either high or low in respect of normal tissue, is indicative of a given outcome, such as survival or recurrence.

### FIELD OF INVENTION

The present invention relates to a method and kit, including parts thereof, for the prognosis of breast cancer. In particular, the method involves identifying a gene expression pattern that indicates the likelihood of survival of a patient with breast cancer and/or likelihood of recurrence of the disease and/or the metastatic character of the cancer in a patient being treated, or having been treated, for breast cancer.

### BACKGROUND OF THE INVENTION

Breast cancer is the most common female cancer in the UK, US and Denmark. It is also the most common form of cancer affecting women in the industrialised world. The incidence of breast cancer has been gradually increasing, and in the US, it is the second most common cause of death due to cancer. Indeed, in 1997, it was estimated that 181,000 new cases were reported in the US, and it has been estimated that 40,000 people die of breast cancer every year. Despite the global efforts that have been made to combat this condition, there has been very little change in the incidence of breast cancer, although early detection and new therapies have marginally improved survival over the past few decades.

While the mechanism of tumorigenesis for most breast carcinomas is largely unknown, there are a number of factors that can predispose some women to developing breast cancer. These include history of birth, menstrual condition, tumour grade, ER status, the size of tumour and the involvement of lymph nodes at the time of diagnosis and surgery. Additionally, prognosis may be determined to varying degrees by the use of mammography or other x-ray imaging methods. However, a mammogram is not Without risk and the breast tumour may be induced by the ionising properties of the radiation used during the test. In addition, such processes are expensive and the results may be interpreted differently by different technicians. For example, one study showed major clinical disagreements in about one third of a set of mammograms that were interpreted by a group of radiologists. Moreover, many women find that undergoing a mammogram is a painful experience.

In clinical practice the prognosis of the disease is important because it determines the treatment that will be given. Accurate prognosis could allow the oncologist to, for example, favour the administration of hormone therapy or chemotherapy and recommend surgery only in the most aggressive cases of cancer.

However, early diagnosis has become a regular feature in breast cancer because more and more patients are now presenting with the disease at a very early stage. This has, made conventional methods of assessing the outcome of the cancer more difficult, and it has become increasingly more evident that it is not only the type of cancer but also the timing of the treatment that is key to how well, or poorly, a patient responds. For example, many patients may currently receive unnecessary treatment that frequently causes toxic side effects, whereas other patients may be put on conservative treatment strategies when in fact a cancer is more advanced than predicted. It can therefore be of vital importance that a correct, and accurate, prognosis is made at an early stage.

To date, no set of satisfactory predictors for prognosis based on clinical information alone has been identified. As a result, research has turned to looking at molecular signatures than can diagnose and prognose cancer. WO 02/103320 discloses thousands of genetic markers whose expression is correlated with clinical prognosis, and which can be used to distinguish patients having good prognoses from poor prognoses. The method for determining expression involves comparing the expression pattern of a test sample of tissue taken from a patient with that of a sample of tissue taken from a patient with a good prognosis and also with that of a sample taken from a patient with a known poor prognosis, and determining which of these samples the test sample most closely corresponds to.

Although this methodology represents an improvement over the traditional clinical methods of prognosis, it does have a number of drawbacks. For example, analysing hundreds of gene markers takes considerable time and is not inherently practical. Furthermore, it is not clear whether a sample that expresses some of the good prognosis markers and some of the bad prognosis markers would give a prognosis of one or the other option. Accordingly, due to the complexity of the methodology, it may either be inaccurate, in the sense that patients are put in the wrong prognosis group because they express more of the genes in one group than the other, or it may be unable to provide a definite answer. As explained previously, early, and accurate, prognosis is vital for appropriate and effective treatment. It is therefore clear that a simpler, more definitive molecular signature is required.

We have therefore developed a method for determining the prognosis of a given breast cancer which is relatively straightforward to perform, efficient to undertake and provides an accurate indication of the likely outcome of the disease. Our method uses a small but highly representative sample of markers which are therefore particularly accurate in determining the likely outcome of a given cancer. Moreover, our method can be divided into three components: a first component that predicts the likely survival of an individual presenting with breast cancer; a second component that predicts the likely recurrence of cancer in an individual presenting with breast cancer; and a third component that predicts the metastatic character of the cancer. As will be apparent to those skilled in the art, the second component therefore indicates the likelihood of incidence free survival of a patient presenting with breast cancer and the third component indicates the aggressive nature of the disease. In summary, we have identified a plurality of molecular signatures that have relevance in determining the prognosis of a given breast cancer. Each molecular signature comprises a plurality of genetic markers whose expression, either high or low in respect of tissue from a patient with moderate prognosis (see hereinafter), is indicative of a given outcome. In addition to this, we have analysed each molecular signature in order to identify which genetic markers are the best indicators of the outcome of a given disease, in other words those that contribute most to the predictive ability of the molecular signature. This subset of markers is known, collectively, as the refined molecular signature.

For example, there is provided a first molecular signature, which comprises two sets of molecular markers whose high expression correlates with low survival rate; the first set comprises those molecular markers that are the most statistically significant indicators of low survival rate, these are referred to herein, collectively, as the first primary molecular signature [Set (A)]:
AMF, ATF4, Cyr61, ER, Matriptase2, MET, MLN64, MMP7, Nectin4, PAR1A, Psoriason, Pttg1, Rho-C, Scotin, SDF1, SEMP1, SPF45, SST1, ST15, TACC2, TBD10, TCF2, TEM6, TEM7R, ZO-3; and
the second set comprises the afore plus at least one of the following molecular markers, referred to herein, collectively, as the first secondary molecular signature [Set (B)]:
Basigin, Beta-catenin, BMP1, BMP10, Calpain large, CD44, CX43, cyclinD2, EHMS, FAK, FAP, GIRK, HAVR1, Isotopo3, JAK1, LOX12, NET-2, PAR1A2, PTHrP, Rho-G, S100A4, SPARC, TCF3, VECAD, Vilip, Wave2.

Reference, herein to the above, and following, markers is reference to a named protein whose full identity is available on the www.NCBI.LM.NIH.gov database or is well known to those skilled in the art.

Reference herein to high or low expression is with respect to the level of expression of the same marker in patients who were deemed to have a moderate prognosis i.e. patients with a standard prognostic index Nottingham Prognosis Index (NPI)=3.4−5.4, where the NPI=0.2×tumour size+tumour grade+nodal status where
NPI (low) is <3.4 and 86% of patients survive 15 years
NPI (moderate) is 3.4−5.4 and 42% of patients survive 15 years
NPI (high) is >5.4 and 13% of patients survive 15 years.

There is further provided a second molecular signature, which comprises two sets of molecular markers whose low expression correlates with low survival rate; the first set comprises those molecular markers that are the most statistically significant Indicators of low survival rate, these are referred to herein, collectively, as the second primary molecular signature [Set (C)]:
ARP2, Atf-3, HuR, MEN1, Paracellin, PTP-RK Radixin, RHO8/gdiG-Ratio; and the second set comprises the afore plus at least one of the following molecular markers, referred to herein, collectively, as the second secondary molecular signature [Set (D)]:
aMOT, AtF-1, Claudin-1, IL22R, Rock 2 Veg1.

There is yet further provided a third molecular signature, which comprises two sets of molecular markers whose high expression correlates with a low incidence of cancer free survival; the first set comprises those molecular markers that are the most statistically significant indicators of a low incidence of cancer free survival, these are referred to herein, collectively, as the third primary molecular signature [Set (E)]:
MMP, AMFR, Bmp9, BMP9, Beta-catenin, CAR, Creb12, DRIM, EHMS, Endomuscin2, FAK, FAP, Isotopo1, Kiss1/ck19. Notch1, PAR1A, Par1A2, PLC-delta, Psoriasin, PTTG1, RhoC, Rock1, SDF₁, SST1, ST15, TEM6, TEM7R; and the second set comprises the afore plus at least one of the following molecular markers, referred to herein, collectively, as the third secondary molecular signature [Set (F)]:
Angiotensin2R1, ATF4, Bmp10, CASM, cathepsinS, CX43, Elastase PMN, GIRK, HAVR1, HIN, Isotopo3, Kiss1, LOX12, NOS3, PMSA, S100A4, SEMP1, TACC2, Ubiquitin, WISP2.

There is further provided a fourth molecular signature, which comprises two sets of molecular markers whose low expression correlates with a low incidence of cancer free survival; the first set comprises those molecular markers that are the most statistically significant indicators of a low incidence of cancer free survival, these are referred to herein, collectively, as the fourth primary molecular signature [Set (G)]:
Bmp3, IL22R, IL24, JAK₁, PTP-RK, Rho8/GdiG, Snail, WASP; and
the second set comprises the afore plus at least one of the following molecular markers, referred to herein, collectively, as the fourth secondary molecular signature [Set (H)]:
ATF3, Bmp4, BMPR1A, MEN1, Paracellin.

There is yet further provided a fifth molecular signature which comprises two sets of molecular markers whose high expression correlates with metastatic cancer; the first set comprises those molecular markers that are the most statistically significant indicators of a metastatic cancer, these are referred to herein, collectively, as the fifth primary molecular signature [Set (I)]:
BAF57, BNDF, CAR1, CASM, Cathepsin-L, Creb1/2, CXCR10, DRIM, HERG, IL7R, IL-11, Kiss1, MKK₁, PMN-elastase, PTTP1, SDF5, TACC2, Ubiquitin, VIPR1, VUDP; and
the second set comprises the afore plus at least one of the following molecular markers referred to herein, collectively, as the fifth secondary molecular signature [Set (J)]:
Angiomotin, BMP7, cyclinD1, DNA ligase-1, IGFBP7, LYVE1, NETZ, RHO8, SRBC, Stath4, TGAse-3, Vinculin, WAVE2.

Finally, there is provided a sixth molecular signature which comprises two sets of molecular markers whose low expression correlates with metastatic cancer; the first set comprises those molecular markers that are the most statistically significant indicators of a metastatic cancer, these are referred to herein, collectively, as the sixth primary molecular signature [Set (K)]:
Paracellin; and
the second set comprises the afore plus at least one of the following molecular markers referred to herein, collectively, as the sixth secondary molecular signature [Set (L)]:
ALCAM, Eplin, ERbeta, Glypic3, JAK1, MAGI-1, PEDF, PKC-eta, Stathlin, WWOX.

We have therefore determined at least six molecular signatures, comprising twelve sets of molecular markers (six primary and six secondary), which have use in the prognosis of breast cancer. The elucidation of these signatures has involved over a decade of work during which time we have systematically and carefully examined hundreds of samples of breast cancer tissue and many more hundreds of genetic molecular markers. However, having completed this arduous task we have, surprisingly, found that, in fact, very few genes need to be examined in order to provide an accurate prognosis for a given sample of breast cancer tissue. Even more surprisingly, we have been able to further reduce this number by identifying those molecular markers that contribute most to the predictive outcome of our molecular signatures, so for example, in the case of the molecular signature relating to metastatic cancer, only 20/21 genes need to be examined. This means that our methodology has immediate application and could be performed quickly and routinely in a clinical context. In fact, we suggest that our methodology forms part of the standard treatment regime of a breast cancer patient so that the relevant oncologist can, at an early stage, determine the outcome of a particular disease and so match the treatment accordingly. Thus, for example, in the case of an individual who presents with a signature indicative of low survival or node metastasis (i.e. the cancer is likely to spread) an immediate and aggressive form of therapy might be prescribed. Similarly, where an individual presents with a signature indicative of low disease-free survival, and therefore is more likely to have a recurrence of the disease, more frequent follow-up visits and tests might be required. Conversely, if an individual has a signature indicative of no metastasis, the oncologist can prescribe less invasive and aggressive treatment, thereby saving the patient from any unnecessary distress and unwanted side effects. Our method therefore not only serves to ensure that individuals receive treatment tailored to their genetic make-up, but it can improve the quality of a patient's life during treatment, by ensuring that aggressive therapy is only prescribed in those cases where it is necessary.

Accordingly, in one aspect of the invention there is provided a method for determining the prognosis of mammalian breast cancer, which method comprises:
(a) examining a sample of breast cancer tissue from an individual in order to determine the expression level of genes encoding the molecular markers in Set (A), and;
(b) where a high level of expression is determined for these markers;
(c) concluding that the individual from whom the tissue sample has been taken has a low likelihood of survival.

In yet a further preferred embodiment of the invention, said methodology, in part (a) thereof, additionally comprises determining the expression level of genes encoding at least one molecular marker in Set (B), in order to determine whether these genes have a high level of expression; and/or the expression level of genes encoding the molecular markers in Set (C), in order to determine whether these genes are under expressed; and/or determining the expression level of genes encoding at least one molecular marker in Set (D), in order to determine whether these genes are under expressed and, if the above expression patterns are identified, concluding that the individual has a low likelihood of survival.

In yet a further aspect of the invention there is provided a method for determining the prognosis of mammalian breast cancer, which method comprises:
(a) examining a sample of breast cancer tissue from an individual in order to determine the expression level of genes encoding the molecular markers in Set (C), and;
(b) where a low level of expression is determined for these markers;
(c) concluding that the individual from whom the tissue sample has been taken has a low likelihood of survival.

In yet a further preferred embodiment of the invention, said methodology, in part (a) thereof, additionally, or alternatively, comprises determining the expression level of genes encoding at least one molecular marker in Set (D) in order to determine whether these genes are under expressed and, if they are, concluding that the individual has a low likelihood of survival.

In yet a further preferred embodiment of this aspect of the invention said methodology, in part (a) thereof, additionally comprises determining the expression level of genes in Set (A) and/or at least one gene in Set (B) in order to determine if these genes are over expressed and, if they are, concluding that the individual has a low likelihood of survival.

In yet a further aspect of the invention there is provided a method for determining the prognosis of mammalian breast cancer, which method comprises:
(a) examining a sample of breast cancer tissue from an individual in order to determine the expression level of genes encoding the molecular markers in Set (E), and;
(b) where a high level of expression is determined for these markers;
(c) concluding that the individual from whom the tissue sample has been taken has a high likelihood of cancer recurrence.

Reference herein to cancer recurrence includes reference to the recurrence of cancer locally, in the breast, or at a remote site or reference to metastasis.

In yet a further preferred embodiment of the invention, the methodology additionally comprises, in part (a) thereof, determining the expression level of genes encoding at least one molecular marker in Set (F), in order to determine whether these genes have a high level of expression; and/or determining the expression level of genes encoding the molecular markers in Set (G), in order to determine whether these genes are under expressed; and/or determining the expression level of genes encoding at least one molecular marker in Set (H), in order to determine whether these genes are under expressed, and if the above expression patterns are identified, concluding that the individual has a high likelihood of cancer recurrence.

In yet a further aspect of the invention there is provided a method for determining the prognosis of mammalian breast cancer, which method comprises:
(a) examining a sample of breast cancer tissue from an individual in order to determine the expression level of genes encoding the molecular markers in Set (G), and;
(b) where a low level of expression is determined for these markers;
(c) concluding that the individuals from whom the tissue sample had been taken has a high likelihood of cancer recurrence.

In yet a further preferred embodiment of the invention, said methodology, in part (a) thereof, additionally, or alternatively, comprises determining the expression level of genes encoding at least one molecular marker in Set (H), in order to determine whether these genes are under expressed and, if they are, concluding that the individual has a high likelihood of cancer recurrence.

In yet a further preferred embodiment of this aspect of the invention said methodology, in part (a) thereof, additionally comprises determining the expression level of genes in Set (E) and/or at least one gene in Set (F) in order to determine if these genes are over expressed and, if they are, concluding that the individual has a high likelihood of cancer recurrence.

In yet a further aspect of the invention there is provided a method for determining the prognosis of mammalian breast cancer, which method comprises:
(a) examining a sample of breast cancer tissue from an individual in order to determine the expression level of genes encoding the molecular markers in Set (I), and;
(b) where a high level of expression is determined for these markers;
(c) concluding that the individual from whom the tissue sample has been taken has a metastatic form of cancer.

In yet a further preferred embodiment of the invention, the methodology additionally comprises, in part (a) thereof, determining the expression level of genes encoding at least one molecular marker in Set (J), in order to determine whether these genes have a high level of expression; and/or determining the expression level of the gene encoding the molecular marker in Set (K), in order to determine whether this gene is under expressed; and/or determining the expression level of genes encoding at least one molecular marker in Set (L), in order to determine whether these genes are under expressed and, if the above expression patterns are identified, concluding that the individual has a metastatic form of cancer.

In yet a further aspect of the invention there is provided a method for determining the prognosis of mammalian breast cancer, which method comprises:
(a) examining a sample of breast cancer tissue from an individual in order to determine the expression level of genes encoding the molecular marker in Set (K), and;
(b) where a low level of expression is determined for this marker;
(c) concluding that the individual from whom the tissue sample has been taken has a metastatic form of cancer.

In yet a further preferred embodiment of the invention, said methodology, in part (a) thereof, additionally, or alternatively, comprises determining the expression level of genes encoding at least one molecular marker in Set (L) in order to determine whether these genes are under expressed and, if they are, concluding that the individual has a metastatic form of cancer.

In yet a further preferred embodiment of this aspect of the invention said methodology, in part (a) thereof, additionally comprises determining the expression level of genes in Set (I) and/or at least one molecular marker in Set (J) in order to determine if these genes are over expressed and, if they are, concluding that the individual has a metastatic form of cancer.

In a further aspect of the invention there is provided any selected combination of all the aforementioned method.

In each of the above methods of the invention, the assay is, ideally, undertaken for human breast cancer tissue and, more preferably still, female human breast cancer tissue.

In each of the above methods of the invention, ideally, the sample of tissue that is examined is assayed for the presence of RNA, preferably total RNA and, more preferably still, the amount of mRNA. It will be apparent to those skilled in the art that techniques available for measuring RNA content are well known and, indeed, routinely practised by those in the clinical diagnostics field.

In an alternative embodiment of the invention the method involves assaying for the protein encoded by each of the molecular markers and so, typically, but not exclusively, involves the use of agents that bind to the relevant proteins and so identify same. Common agents are antibodies and, most ideally, monoclonal antibodies which, advantageously, have been labelled with a suitable tag whereby the existence of the bound antibody can be determined. Assay techniques for identifying proteins are well known to those skilled in the art and indeed used every day by workers in the field of clinical diagnostics.

Additionally, the methodology of the invention may involve the amplification of a selected marker prior to the identification of same and in this case, typically, amplification will be undertaken using a PCR reaction wherein oligonucleotide probes specific for the molecular marker of interest are used in order to amplify same prior to determining the presence and, having regard to the degree of amplification, the amount thereof.

In further preferred methods of working the invention the level of expression of a given molecular marker is determined having regard to a control sample, wherein the control sample is a sample of breast tissue which is cancer free or from a patient with a moderate prognosis as herein defined. More ideally still this sample of breast tissue is taken from an individual who is not presenting with the disease. Alternatively still, the control is a recognised standard for expression of each relevant gene in a healthy individual.

The level of gene expression may be measured by real-time quantitative PCR, using a method disclosed in Jiang et al 2003a or Parr and Jiang 2004.

The likelihood of survival means the likelihood that the patient will be alive for the next 10 years. The likelihood of recurrence means the likelihood that the cancer will recur within 10 years. A metastatic form of cancer means that the cancer will have spread from the organ or tissue of origin to another part of the body.

According to yet a further aspect of the invention there is provided a kit for performing any one or more of the aforementioned methods wherein said kit comprises:
(a) a plurality of probes for detecting at least one Set of the molecular markers specified in the aforementioned methods; and
(b) optionally, reagents and instructions pertaining to the use of said probes.

In yet a further preferred aspect of the invention there is provided a kit for determining the prognosis of mammalian breast cancer which comprises:
(a) a plurality of probes for identifying at least one transcript of each of the genes in Set (A), and;
(b) optionally, reagents and instructions that determine, or show how to determine, the level of expression of each of said genes.

In yet a further embodiment of the invention, said kit additionally comprises:
(a) a plurality of probes for identifying: at least one transcript of each of the genes in Set (C), and/or at least one transcript for at least one of the genes in Set (B) or (D), and;
(b) optionally, reagents and instructions that determine, or show how to determine, the level of expression of each of said genes.

In yet a further preferred aspect of the invention there is provided a kit for determining the prognosis of mammalian breast cancer which comprises:
(a) a plurality of probes for identifying at least one transcript of each of the genes in Set (E), and;
(b) optionally, reagents and instructions that determine, or show how to determine, the level of expression of each of said genes.

In yet a further preferred aspect of the invention said kit additionally comprises:
(a) a plurality of probes for identifying: at least one transcript of each of the genes in Set (G) and/or at least one transcript for at least one of the genes in Set (F),or (H), and;
(b) optionally, reagents and instructions that determine, or show how to determine, the level of expression of each of said genes.

In yet a further preferred aspect of the invention there is provided a kit for determining the prognosis of mammalian breast cancer which comprises:
(a) a plurality of probes for identifying at least one transcript of each of the genes in Set (I), and;
(b) optionally, reagents and instructions that determine, or show how to determine, the level of expression of each of said genes.

In yet a further preferred aspect of the invention said kit additionally comprises:
(a) a plurality of probes for identifying: at least one transcript of each of the genes in Set (K), and/or at least one transcript for at least one of the genes in Set (J) or (L), and;
(b) optionally, reagents and instructions that determine, or show how to determine, the level of expression of each of said genes.

In a further aspect of the invention there is provided a kit comprising any selected combination of the aforementioned sets of probes for identifying the aforementioned sets of molecular markers.

According to yet further aspect of the invention there is provided a microarray comprising any one or more of the aforementioned sets of probes for identifying the level of expression of any one or more of the aforementioned sets of molecular markers.

In another aspect of the invention, there is provided a kit for determining the likelihood of survival and/or recurrence of breast cancer and/or metastatic nature of a cancer in a patient, which kit comprises:
(a) at least one microarray comprising a plurality of probes for identifying at least one set of the molecular markers described in the above methods; and, optionally,
(b) a second microarray comprising a plurality of probes for identifying the same set of molecular markers in an internal standard that represents the normal level of expression of said markers.

The invention also provides a microarray or set of probes as described above.

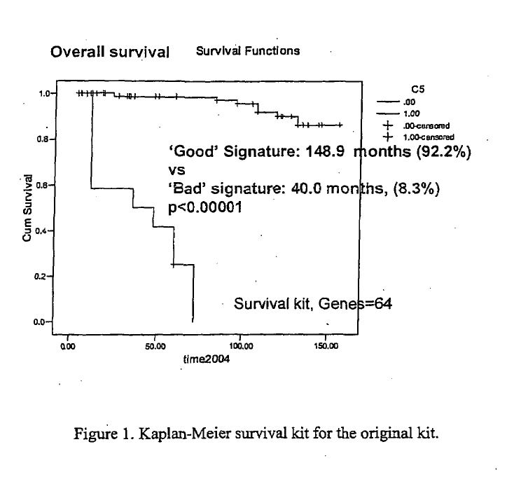

The present invention will now be described by way of the following examples with reference to Tables 1-3 and FIGS. 1-4 wherein:

FIG. 1 shows a Kaplan-Meier survival curve for all the markers in Table 1.

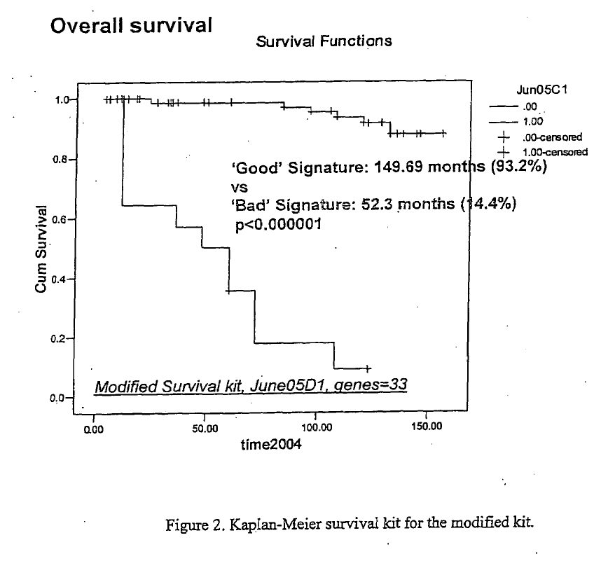

FIG. 2 shows a Kaplan-Meier survival curve for markers indicated with a * in Table 1.

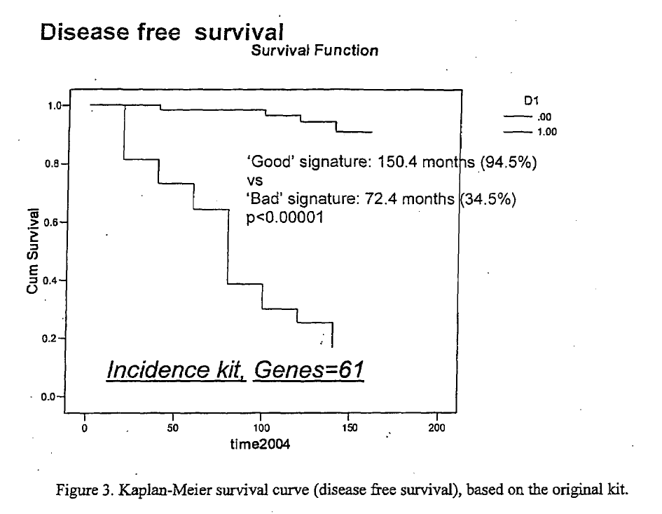

FIG. 3 shows a Kaplan-Meier survival curve for all the markers in Table 2.

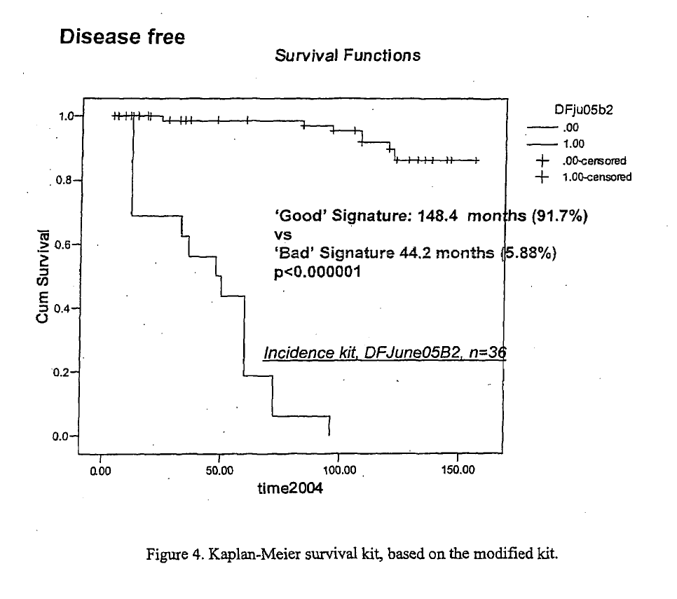

FIG. 4 shows a Kaplan-Meier survival curve for markers indicated with a *in Table 2.

### TISSUES AND CELLS

Breast tumour tissues and associated normal tissues were collected immediately after surgery and frozen until use. This was under the approval of a local ethical committee and took place mainly between 1991-1994, with limited number collected between 1995-1996. The current analysis is based on a median follow up of 10 years as at June 2004. The current study has used breast cancer tissues (n=120) and normal background tissues (n=32). Human breast cancer cell lines MCF-7 and MDA MB 231, human fibroblast cell line MRC-5 were purchased from the European Collection of Animal Cell Cultures (ECACC, Salisbury, England). Human umbilical vein endothelial cells (HUVEC) were purchased from TCS Biologicals (Oxford, England). Information on the pathology, clinical information during and after surgery, patient clinical outcomes were obtained soon after surgery or at the time of follow up.

### Tissues Processing

Mammary tissues were frozen sectioned. Sections were divided into the following three parts: one portion for routine histology, one portion for immunohistochemistry and the other portion was for preparation of RNA.

Extraction of RNA from Cells and Tissues and cDNA Synthesis

Frozen sections of tissues were cut at a thickness of 5-10 μm and were kept for immunohistochemistry and routine histology (Jiang et al 2003a). A further 15-20 sectioris were homogenised using a hand-held homogeniser, in ice-cold RNA extraction solution. The concentration of RNA was determined using a UV spectrophotometer. Reverse transcription was carried using a RT kit with an anchored oligo-dt primer supplied by AbGene™, using 1 μg total RNA in 96-well plate. The quality of cDNA was verified using β-actin primers. RNA extraction kit and RT kit were obtained from AbGene Ltd, Surrey, England, UK. PCR primers were designed using Beacon Designer (California, USA) and synthesised by Invitrogen™ Ltd (Paisley, Scotland, UK). Molecular biology grade agarose and DNA ladder were from Invitrogen. Mastermix for routine PCR and quantitative PCR was from AbGene.

### Quantitative Analysis of Genetic Markers

The transcript level of the CCN family members from the above-prepared cDNA was determined using a real-time quantitative PCR, based on the Amplifuor™ technology (Nazarenko et al 1997), modified from a method previous reported (Jiang et al 2003a and 2003b). Briefly, a pair of PCR primers were designed using the Beacon Designer software (version 2, California, USA). To one of the primers (routinely to the antisense primer in our laboratory), an additional sequence, known as the Z sequence (5′ actgaacctgaccgtaca′3) which is complementary to the universal Z probe (Nazarenko et at 1997) (Intergen Inc., England, UK), was added. A Taqman™ detection kit for β-actin was purchased from Perkin-Elmer™.

The reaction was carried out using the following: Hot-start Q-master mix (Abgene), 10 pmol of specific forward primer, 1 pmol reverse primer which has the Z sequence, 10 pmol of FAM-tagged probe (Intergen Inc.), and cDNA from approximate 50 ng RNA (calculated from the starting RNA in the RT reaction). The reaction was carried out using IcyclerlQ™ (Bio-Rad™) which is equipped with an optic unit that allows real time detection of 96 reactions, using the following condition: 94° C. for 12 minutes, 50 cycles of 94° C. for 15 seconds, 55° C. for 40 seconds and 72° C. for 20 seconds (Jiang et al 2003b, 2003c, Parr and Jiang 2004). The levels of the transcripts were generated from an internal standard (Jiang et al 2003a) that was simultaneously amplified with the samples. The results are shown here in two ways: levels of transcripts based on equal amounts of RNA, or as a target/CK19 ratio.

Immunohistochemical Staining of the Molecules where Appropriate

Frozen sections of breast tumour and background tissue were cut at a thickness of 6 μm using a cryostat (Jiang et al 2003c). The sections were mounted on super frost plus microscope slides, air dried and then fixed in a mixture of 50% acetone and 50% methanol. The sections were then placed in “Optimax” wash buffer for 5-10 minutes to rehydrate. Sections were incubated for 20 mins in a 0.6% BSA blocking solution and probed with a primary antibody. Following extensive washings, sections were incubated for 30 minutes in a secondary biotinylated antibody (Multilink Swine anti-goat/mouse/rabbit immunoglobulin, Dako Inc.). Following washings, Avidin Biotin Complex (Vector Laboratories) was then applied to the sections followed by extensive washings. Diaminobenzidine chromogen (Vector Labs) was then added to the sections which were incubated in the dark for 5 minutes. Sections were then counter stained in Gill's Haematoxylin and dehydrated in ascending grades of methanol before clearing in xylene and mounting under a cover slip. Cytoplasmic staining of the respective proteins was quantified using Optimas 6.0 software as we previously described (Davies et al 2000, King et al 2004) and is shown here as relative staining intensity.

### Statistical Analysis

Statistical analysis was carried out using Mann-Whitney U test and the Kruskal-Wallis test. Survival analysis was carried out using Kaplan-Meier survival curve and Univariate analysis (SPSS11).

### Results

### Molecules Screened

We have quantified 453 molecules against the full clinical information that includes a 10-year follow up. Following analysis on survival rates and incidence of recurrence of the disease, we have developed three signatures, the survival molecular signature and the incidence prediction molecular signature and the metastatic molecular signature.

### The Survival Molecular Signature

As shown in Table 1, 51 molecules were found to have a positive correlation with low survival and 14 inversely correlated with low survival. FIG. 1 shows that 92.2% of individuals having what is termed herein as a “good signature” (that is not having a high expression of the molecules in the left-hand column of Table 1, and not having under expression of the molecules in the right-hand column of Table 1), are predicted to survive for up to 148.9 months, while only 8.3% of individuals having what is termed herein as a “bad signature” (that is having a high expression of the molecules in the left-hand column of Table 1, and under expression of the molecules in the right-hand column of Table 1) are predicted to survive for up to 40 months. This result is statistically significant, with a p value <0.00001.

Using the Kaplan-Meier survival curve and univariate analysis, we refined the molecular signature by identifying those molecules that contribute most to the statistical accuracy (identified by * in Table 1). We have found that 33 primary molecular markers, 25 of which have a positive correlation with low survival and 8 of which have a negative correlation with low survival, account for the majority of the statistical significance. FIG. 2 shows the predicted survival curve using the first primary and second primary molecular signature, with 93.2% of individuals having a good signature predicted to survive for up to 149.69 months, and only 14.4% of individuals having a bad signature predicted to survive for up to 52.3 months (p<0.000001).

### The Incidence Free Prediction Molecular Signature

As shown in Table 2, 48 molecules were found to have a positive correlation with occurrence of incidence (recurrence and metastasis) and 13 inversely correlated with occurence of incidence. FIG. 3 shows that 94.5% of individuals having a good signature (that is not having a high expression of the molecules in the left-hand column of Table 2, and not having under expression of the molecules in the right-hand column of Table 2) are predicted to have no recurrence of the disease (i e. disease-free survival) for up to 150.4 months, while only 34.5% of individuals having a bad signature (that is having a high expression of the molecules in the left-hand column of Table 2, and under expression of the molecules in the right-hand column of Table 2) are predicted to live disease-free for only up to 72.4 months (p<0.00001).

As with the survival signature above, we also refined this signature, and found that 36 primary molecular markers (indicated by * in Table 2), 28 of which have a positive correlation with recurrence, and 8 of which have a negative correlation with recurrence account for the majority of the statistical significance. FIG. 4 shows the predicted survival curve using the third primary and fourth primary molecular signature, with 91.7% of individuals having a good signature predicted to have no recurrence of the disease for up to 148.4 months, and only 5.88% of individuals having a bad signature predicted to survive, without any recurrence of the disease, for up to 44.2 months (p<0.000001).

### The Molecular Signature of Node Metastasis

As shown in Table 3, 37 molecules were found to have a positive correlation with nodal metastasis and 10 inversely correlated with node metastasis. The combination of these 37 molecules has shown that 91% of tumours with a bad signature (that is having a high expression of the molecules in the left-hand column of Table 3, and under expression of the molecules in the right-hand column of Table 3) developed node metastasis. Furthermore, 88.9% of tumours with a good signature (that is not having a high expression of the molecules in the left-hand column of Table 3,and not having under expression of the molecules in the right-hand column of Table 3) had no node metastasis (p=0.00024).

As above, we have modified this signature by refining the combination, and found that a combination of 21 primary genes, 20 of which are positively correlated with node metastasis and 1 of which is negatively correlated with node metastasis (indicated by * in Table 3), also predicted well. 89.1% of tumours with a bad signature had node metastasis and 86.8% of tumours with a good signature had no node metastasis (p=0.0000205).

### REFERENCES

Davies et al 2000: Davies G, Jiang W G, Mason M D. Cell-cell adhesion and signalling intermediates in human prostate cancer. Journal of Urology, 2000, 163, 985-992
Jiang et al 2003a: Jiang W G, Watkins G, Lane J, Douglas-Jones A, Cunnick G H, Mokbel M, Mansel R E. Prognostic value of Rho family and and rho-GDIs in breast cancer. Clinical Cancer Research, 2003, 9 (17), 6432-6440
Jiang et al 2003b: Jiang W G, Douglas-Jones A, and Mansel R E. Level of expression of PPAR-gamma and its co-activator (PPAR-GCA) in human breast cancer.
International Journal of Cancer, 2003, 106, 752-757
Jiang et al 2003c: Jiang W G, Grimshaw D, Lane J, Martin T A, Parr C, Davies G, Laterra J, and Mansel R E. Retroviral hammerhead transgenes to cMET and HGF/SF inhibited growth of breast tumour, induced by fibroblasts. Clinical Cancer Research, 2003, 9, 4274-4281
King et al 2004: King J A C, Ofori-Acquah A F, Stevens T, Al-Mehdi A B, Fodstad O; Jiang W G. Prognostic value of ALCAM in human breast cancer. Breast Cancer Research, 2004, R478-487
Nazarenko et al 1997: Nazarenko I A, Bhatnagar S K, Hohman R J. A closed tube format for amplification and detection of DNA based on energy transfer. Nucleic Acids Res 1997;25: 2516-21
Parr and Jiang 2004: Parr C and Jiang W G. The Notch receptors, Notch-1 and Notch-2, in human breast cancers. International Journal of Molecular Medicine, 2004 Nov.;14(5): 779-786

TABLE 1
Molecular signature for overall survival

| High with incidence | | | |
|---|---|---|---|
| | Modified kit | Low with incidence | |
| Original kit | (genes (indicated | | Modified kit |
| (survival curve | by *, survival | Original kit | (genes (indicated |
| is FIG. 1) | curve is FIG. 2) | (FIG. 1) | by *, FIG. 2) |
| AMF | * | aMOT | |
| ATF4 | * | ARP2 | * |
| Basigin | | Atf-1 | |
| Beta-catenin | | Atf-3 | * |
| BMP1 | | Claudin-1 | |
| BMP10 | | HuR (0.05) | * |
| Calpain large | | IL22R | |
| CD44 | | MEN1 (0.02) | * |
| CX43 | | Paracellin | * |
| cyclinD2 | | PTP-RK | * |
| Cyr61 | * | Radixin | * |
| EHMS | | RHO8/gdiG-Ratio | * |
| ER | * | Rock 2 | |
| FAK | | VEG1 | |
| FAP | | | |
| GIRK | | | |
| HAVR1 | | | |
| Isotopo3 | | | |
| JAK1 | | | |
| LOX12 | | | |
| Matriptase2 | * | | |
| MET | * | | |
| MLN64 | * | | |
| MMP7 | * | | |
| Nectin4 | * | | |
| NET-2 | | | |
| PAR1A | * | | |
| PAR1A2 | | | |
| Psoriason | * | | |
| PTHrP | | | |
| Pttg1 | * | | |
| Rho-C | * | | |
| Rho-G | | | |
| S100A4 | | | |
| Scotin | * | | |
| SDF1 | * | | |
| SEMP1 | * | | |
| SPARC | | | |
| SPF45 | * | | |
| SST1 | * | | |
| ST15 | * | | |
| TACC2 | * | | |
| TBD10 | * | | |
| TCF2 | * | | |
| TCF3 | | | |
| TEM6 | * | | |
| TEM7R | * | | |
| VECAD | | | |
| Vilip | | | |
| Wave2 | | | |
| ZO-3 | * | | |

TABLE 2
Molecular signature for incidence free survival in human breast cancer

| High with incidence | | Low with incidence | |
|---|---|---|---|
| Original kit | Modified kit | | Modified kit |
| (survival curve | (genes (indicated | Original kit | (genes (indicated |
| is FIG. 3) | by *, FIG. 4) | (FIG. 3) | by *, FIG. 4) |
| AAMP | * | ATF3 | |
| AMFR | * | Bmp3 | * |
| Angiotensin2R1 | | Bmp4 | |
| ATF4 | | BMPR1A | |
| Bmp8 | * | IL22R | * |
| BMP9 | * | IL24 | * |
| Bmp10 | | JAK1 | * |
| Beta-catenin | * | MEN1 | |
| CAR | * | Paracellin | |
| CASM | | PTP-RK | * |
| cathepsinS | | Rho8/GdiG | * |
| Creb12 | * | Snail | * |
| CX43 | | WASP | * |
| DRIM | * | | |
| EHMS | * | | |
| Elastase PMN | | | |
| Endomuscin2 | * | | |
| FAK | * | | |
| FAP | * | | |
| GIRK | | | |
| HAVR1 | | | |
| HIN | | | |
| Isotopo1 | * | | |
| Isotopo3 | | | |
| Kiss1 | | | |
| Kiss1/ck19 | * | | |
| LOX12 | | | |
| NOS3 | | | |
| Notch1 | * | | |
| PAR1A | * | | |
| Par1A2 | * | | |
| PLC-delta | * | | |
| PMSA | | | |
| Psoriasin | * | | |
| PTTG1 | * | | |
| RhoC | * | | |
| Rock1 | * | | |
| S100A4 | | | |
| SDF1 | * | | |
| SEMP1 | | | |
| SST1 | * | | |
| ST15 | * | | |
| TACC2 | | | |
| TEM6 | * | | |
| TEM7R | * | | |
| Ubiquitin | | | |
| WISP2 | | | |

Original kit, genes = 61
Modified kit, genes = 36

TABLE 3
Molecular signature for predicting node metastasis

| Significantly high with node metastasis | | Signficiantly low with incidence | |
|---|---|---|---|
| | Modified | | Modified |
| Initial filing | signature(*) | Initial filing | signature (*) |
| Angiomotin | | ALCAM | |
| BAF57 | * | Eplin | |
| BMP7 | | ERbeta | |
| BNDF | * | Glypic3 | |
| CAR1 | * | JAK1 | |
| CASM | * | MAGI-1 | |
| Cathepsin-L | * | Paracellin | * |
| Creb1/2 | * | PEDF | |
| CXCR10 | * | PKC-eta | |
| cyclinD1 | | Stathlin | |
| DNA ligase-1 | | WWOX | |
| DRIM | * | | |
| HERG | * | | |
| IGFBP7 | | | |
| IL7R | * | | |
| IL-11 | * | | |
| Kiss1 | * | | |
| LYVE1 | | | |
| MKK1 | * | | |
| NET2 | | | |
| PMN-elastase | * | | |
| PTTP1 | * | | |
| RHO8 | | | |
| SDF5 | * | | |
| SRBC | | | |
| Stath4 | | | |
| TACC2 | * | | |
| TGAse-3 | | | |
| Ubiquitin | * | | |
| Vinculin | | | |
| VIPR1 | * | | |
| VUDP | * | | |
| WAVE2 | | | |

1. A method for determining the prognosis of mammalian breast cancer, which method comprises:
(a) examining a sample of breast cancer tissue from an individual in order to determine the expression level of geries encoding the following molecular markers: AMF, ATF4, Cyr61, ER, Matriptase2, MET, MLN64, MMP7, Nectin4, PAR1A, Psoriason, Pttg1, Rho-C, Scotin, SDF1, SEMP1, SPF45, SST1, ST15, TACC2, TBD10, TCF2, TEM6, TEM7R, ZO-3; and
(b) where a high level of expression is determined for these markers;
(c) concluding that the individual from whom the tissue sample has been taken has a low likelihood of survival.

2. A method according to claim 1, wherein part (a) additionally comprises determining the expression level of genes encoding at least one of the following molecular markers: Basigin, Beta-catenin, BMP1, BMP10, Calpain large, CD44, CX43, cyclinD2, EHMS, FAK, FAP, GIRK, HAVR1, Isotopo3, JAK1, LOX12, NET-2, PAR1A2, PTHrP, Rho-G, S100A4, SPARC, TCF3, VECAD, Vilip, Wave2.

3. A method according to claim 1 or claim 2, wherein part (a) additionally comprises determining the expression level of genes encoding the following molecular markers: ARP2, Aff-3, HuR, MEN1, Paracellin, PTP-RK Radixin, RHO8/gdiG-Ratio; and
(b) where a low level of expression is determined for these markers;
(c) concluding that the individual from whom the tissue sample has been taken has a low likelihood of survival.

4. A method according to claim 3, wherein part (a) additionally comprises determining the expression level of genes encoding at least one of the following molecular markers: aMOT, Atf-1, Claudin-1, IL22R, Rock 2, Veg1.

5. A method for determining the prognosis of mammalian breast cancer, which method comprises:
(a) examining a sample of breast cancer tissue from an individual in order to determine the expression level of genes encoding the following molecular markers: AAMP, AMFR, Bmp8, BMP9, Beta-catenin, CAR, Creb12, DRIM, EHMS, Endomuscin2, FAK, FAP, Isotopo1, Kiss1/ck19, Notch1, PAR1A, Par1A2, PLC-delta, Psoriasin, PTTG1, RhoC, Rock1, SDF1, SST1, ST15, TEM6, TEM7R; and
(b) where a high level of expression is determined for these markers;
(c) concluding that the individual from whom the tissue sample has been taken has a high likelihood of cancer recurrence.

6. A method according to claim 5, wherein part (a) additionally comprises determining the expression level of genes encoding at. least one of the following molecular markers: Angiotensin2R1, ATF4, Bmp10, CASM, cathepsinS, CX43, Elastase PMN, GIRK, HAVR1, HIN, Isotopo3, Kiss1, LOX12, NOS3, PMSA, S100A4, SEMP1, TACC2, Ubiquitin, WISP2.

7. A method according to claim 5 or claim 6, wherein part (a) additionally comprises determining the expression level of genes encoding the following molecular markers: Bmp3, IL22R, IL24, JAK1, PTP-RK, Rho8/GdiG, Snail, WASP, and;
(b) where a low level of expression is determined for these markers:
(c) concluding that the individual from whom the tissue sample has been taken has a high likelihood of cancer recurrence.

8. A method according to claim 7, wherein part (a) additionally comprises determining the expression level of genes encoding at least one of the following molecular markers: ATF3, Bmp4, BMPR1A, MEN1, Paracellin.

9. A method for determining the prognosis of mammalian breast cancer, which method comprises:
(a) examining a sample of breast cancer tissue from an individual in order to determine the expression level of genes encoding the following molecular markers: BAF57, BNDF, CAR1, CASM, Cathepsin-L, Creb1/2, CXCR10, DRIM, HERG, IL7R, IL-11, Kiss1, MKK1, PMN-elastase, PTTP1, SDF5, TACC2, Ubiquitin, VIPR1, VUDP; and
(b) where a high level of expression is determined for these markers;
(c) concluding that the individual from whom the tissue sample has been taken has a metastatic form of cancer.

10. A method according to claim 9, wherein part (a) additionally comprises determining the expression level of genes encoding at least one of the following molecular markers: Angiomotin, BMP7, cyclinD1, DNA ligase-1, IGFBP7, LYVE1, NET2, RHO8, SRBC, Stath4, TGAse-3, Vinculin, WAVE2.

11. A method according to claim 9 or claim 10, wherein part (a) additionally comprises determining the expression level of the gene encoding the molecular marker: Paracellin; and
(b) where a low level of expression is determined for this marker;
(c) concluding that the individual from whom the tissue sample has been taken has a metastatic form of cancer.

12. A method according to claim 11, wherein part (a) additionally comprises determining the expression level of genes encoding at least one of the following molecular markers: ALCAM, Eplin, ERbeta, Glypic3, JAK1, MAGI-1, PEDF, PKC-eta, Stathlin, WWOX.

13. A method according to any preceding claim, wherein the cancer tissue is from a human.

14. A method according to any preceding claim, wherein the cancer tissue is from a female.

15. A method according to any preceding claim, wherein the level of expression is determined by assaying for the presence of RNA or mRNA.

16. A method according to any one of claims 1-15, wherein the level of expression is determined by assaying for the protein(s) encoded by the molecular markers.

17. A method according to claim 16, wherein the method involves the use of agents that bind to the relevant protein(s) and so identify same.

18. A method according to claim 17, wherein the agents are antibodies.

19. A method according to any of claims 1-14, wherein prior to performing part (a), the selected marker is amplified.

20. A method according to claim 19, wherein the marker is amplified by PCR.

21. A method according to any preceding claim, wherein the level of expression of a given molecular marker is determined having regard to a control sample, wherein the control sample is any one of the following: a sample of breast tissue which is cancer free, a sample of breast tissue taken from an individual who is not presenting with cancer, or a recognised standard for expression of each relevant molecular marker in a healthy individual.

22. A kit for performing a method according to any one of claims 1-21 wherein said kit comprises:
(a) a plurality of probes for detecting at least one Set of the molecular markers specified in the method of claims 1-21; and
(b) optionally, reagents and instructions pertaining to the use of said probes.

23. A kit for determining the prognosis of mammalian breast cancer which comprises:
(a) a plurality of probes for identifying at least one transcript of each of the genes in the following set of markers: AMF, ATF4, Cyr61, ER, Matriptase2, MET, MLN64, MMP7, Nectin4, PAR1A, Psoriason, Pttg1, Rho-C, Scotin, SDF1, SEMP1, SPF45, SST1, ST15, TACC2, TBD10, TCF2, TEM6, TEM7R, ZO-3; and
(b) optionally, reagents and instructions that determine, or show how to determine, the level of expression of each of said genes.

24. A kit according to claim 23, wherein said kit additionally comprises:
(a) a plurality of probes capable of identifying at least one transcript of at least one of the genes in the following set of markers: Basigin, Beta-catenin, BMP1, BMP10, Calpain large, CD44, CX43, cyclinD2, EHMS, FAK, FAP, GIRK, HAVR1, Isotopo3, JAK1, LOX12, NET-2, PAR1A2, PTHrP, Rho-G, S100A4, SPARC, TCF3, VECAD, Vilip, Wave2, and/or at least one transcript of each of the genes in the following'set of markers ARP2, Aff-3, HuR, MEN1, Paracellin, PTP-RK Radixin, RHO8/gdiG-Ratio, and/or at least one transcript of at least one of the following set df-markers: aMOT, Aff-1, Claudin-1, IL22R, Rock 2, and;
(b) optionally, reagents and instructions that determine the level of expression of each of said genes.

25. A kit for determining the prognosis of mammalian breast cancer which comprises:
(a) a plurality of probes for identifying at least one transcript of each of the genes in the following set of markers: AMFR, AAMP, Beta-catenin, Bmp8, BMP9, CAR, Creb12, DRIM, EHMS, Endomuscin2, FAK, FAP, Isotopo1, Kiss1/ck19, Notch1, PAR1A, Par1A2, PLC-delta, Psoriasin, PTTG1, RhoC, Rock1, SDF1, ST15, SST1, TEM6, TEM7R; and
(b) optionally, reagents and instructions that determine, or show how to determine, the level of expression of each of said genes.

26. A kit according to claim 25, wherein the kit additionally comprises:
(a) a plurality of probes for identifying at least one transcript of at least one of the genes in the following set of markers: Angiotensin2R1, ATF4, Bmp10, CASM, cathepsinS, CX43, Elastase PMN, GIRK, HAVR1, HIN, Isotopo3,Kiss1, LOX12, NOS3, PMSA, S100A4, SEMP1, TACC2, Ubiquitin, WISP2 and/or at least one transcript of each of the genes of the following set of markers: Bmp3, IL22R, IL24, JAK1, PTP-RK, Rho8/GdiG, Snail, WASP, and/or at least one transcript of at least one of the following set of markers: ATF3, Bmp4, BMPR1A, MEN1, Paracellin: and
(b) optionally, reagents and instructions that determine, or show how to determine, the level of expression of each of said genes.

27. A kit for determining the prognosis of mammalian breast cancer which comprises:
(a) a plurality of probes for identifying at least one transcript of each of the genes in the following set of markers: BAF57, BNDF, CAR1, CASM, Cathepsin-L, Creb1/2, CXCR10, DRIM, HERG, IL7R, IL-11, Kiss1, MKK1, PMN-elastase, PTTP1, SDF5, TACC2, VIPR1, VUDP, Ubiquitin; and
(b) optionally, reagents and instructions that determine, or show how to determine, the level of expression of each of said genes.

28. A kit according to claim 27, wherein said kit additionally comprises:
(a) a plurality of probes for identifying at least one transcript of at least one of the genes in the following set of markers: Angiomotin, BMP7, cyclinD1, DNA ligase-1, IGFBP7, LYVE1, NET2, RHO8, SRBC, Stath4, TGAse-3, Vinculin, WAVE2, and/or the following set of markers: Paracellin, and/or at least one transcript of at least one of the following set of markers: ALCAM, Eplin, ERbeta, Glypic3, JAK1, MAGI-1, PEDF, PKC-eta, Stathlin, WWOX; and
(b) optionally, reagents and instructions that determine, or show how to determine, the level of expression of each of said genes.

29. A microarray comprising at least one set of probes for identifying the sets of molecular markers that comprise the molecular signatures described in claims 22-28.

30. A kit for determining the likelihood of survival and/or recurrence of breast cancer and/or the metastatic nature of a cancer in a patient, which kit comprises:
(a) at least one microarray comprising at least one set of probes for identifying the sets of molecular markers that comprise the molecular signatures described in claims 1-21; and, optionally,
(b) a secondary microarray comprising a plurality of probes for identifying the same set of molecular markers in an internal standard that represents the level of expression of said markers in either a cancer free individual or a patient with a moderate prognosis.

31. A microarray according to claim 30.

32. A set of probes according to claims 22-28.

33. A method, kit or parts thereof, as substantially herein described.
